(19) United States
(12) Patent Application Publication
(10) Pub. No.: US 2012/0308634 A1
(43) Pub. Date: Dec. 6, 2012

(54) GUM DELIVERY SYSTEM

(76) Inventor: Brent Holland, Covington, LA (US)

(21) Appl. No.: 13/527,085

(22) Filed: Jun. 19, 2012

Related U.S. Application Data
(63) Continuation of application No. 12/962,459, filed on Dec. 7, 2010, now abandoned.
(60) Provisional application No. 61/267,682, filed on Dec. 8, 2009.

(51) Int. Cl. A23G 4/20; A61P 39/06; A23L 1/03; A61K 9/68

(52) U.S. Cl. 424/440; 424/48; 426/5

(57) ABSTRACT

A new chewing gum comprising a gum member, with a central core or cavity, and a core element located within the central core or cavity. The chewing gum also comprises a dietary supplement, which may be incorporated in the central core, the cavity, or both. The core element can be of a range of consistencies, from fluid to gel-like. Optionally, the gum member may comprise crystals. Additionally, the crystals may comprise a dietary supplement. The chewing gum may be encapsulated with a coating. Multiple dietary supplements, of different or of the same varieties, may be incorporated into the chewing gum.

### CROSS REFERENCE TO RELATED APPLICATIONS

This application is a continuation of and claims priority to U.S. application Ser. No. 12/962,459, filed on Dec. 7, 2010, which is a non-provisional of and claims priority to U.S. application Ser. No. 61/267,682, filed on Dec. 8, 2009.

### STATEMENT REGARDING FEDERALLY SPONSORED RESEARCH OR DEVELOPMENT

Not Applicable.

### REFERENCE TO A “SEQUENCE LISTING,” A TABLE, OR A COMPUTER PROGRAM

Not Applicable.

### DESCRIPTION OF THE DRAWINGS

FIG. 1 is a cut away view of a new chewing gum.

FIG. 2 depicts a cut away view of an alternate embodiment of the chewing gum.

FIG. 3 depicts a cut away view of an alternate embodiment of the chewing gum.

FIG. 4 depicts a cut away view of an alternate embodiment of the chewing gum.

### DETAILED DESCRIPTION OF THE INVENTION

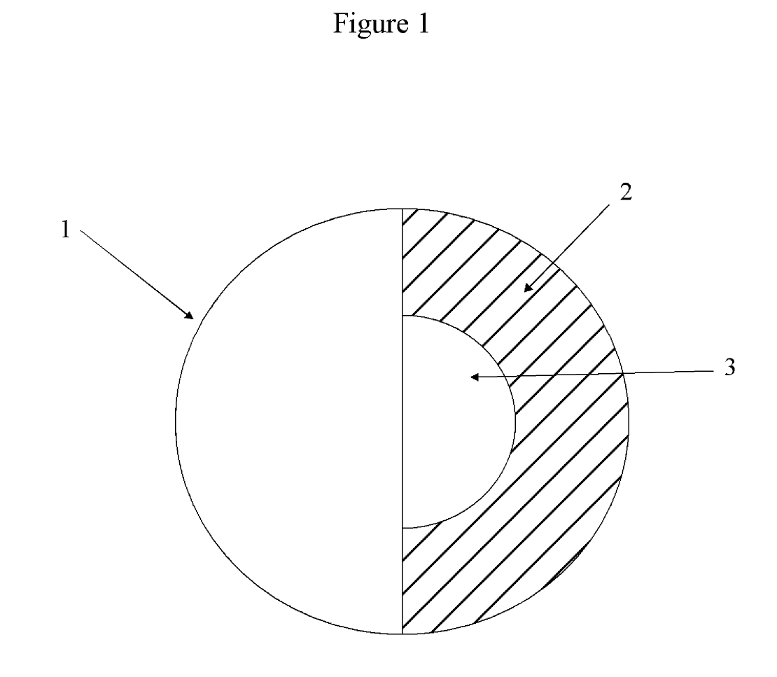

With reference to FIG. 1, a new chewing gum, 1, is disclosed wherein a dietary supplement is incorporated into the chewing gum. As used herein, the term dietary supplement means any substance which is beneficial to the health of a human being. A non-exclusive list of dietary supplements includes appetite suppressants, natural energizers, vitamins, minerals, and antioxidants. As used herein, the term appetite suppressant means any known appetite suppressant which is capable of providing an appetite suppressant effect when ingested by a person and any combination thereof. Also, as used herein, the term “natural energizer” means any known energizer derived from natural sources which is capable of providing an increase in energy to a person after ingestion and any combination thereof. The dietary supplement or combinations of dietary supplements may be incorporated throughout the entire chewing gum composition or only in portion thereof. Also, different combinations of dietary supplements may be incorporated into different aspects of the gum composition.

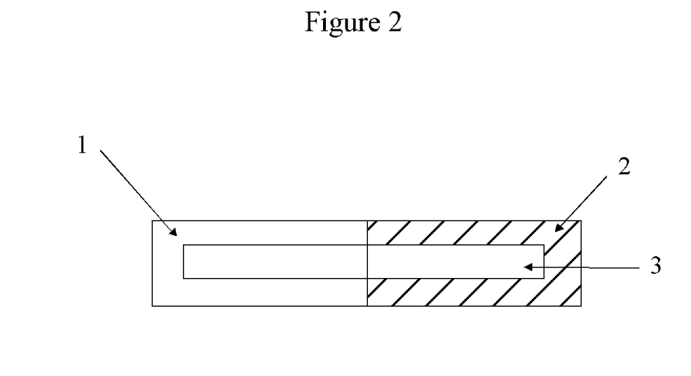

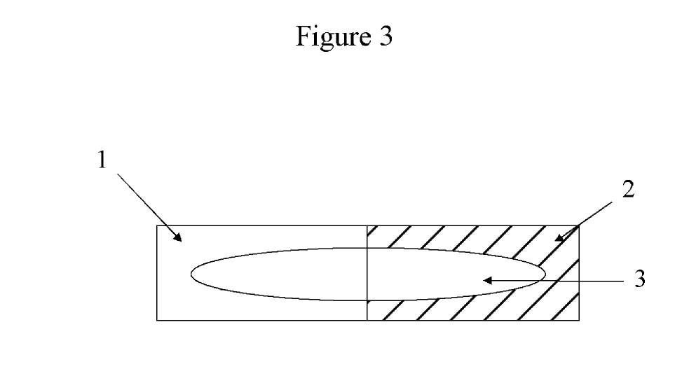

The chewing gum, 1, with a dietary supplement incorporated into the gum base generally comprises a gum member, 2, having a central core or cavity, 3. The gum member, 2, may be any shape of gum, including but not limited to a gum ball (shown in FIG. 1), an elongate gum, as depicted in FIGS. 2 and 3, or a square shape (not shown). A liquid center comprising a dietary supplement is located within the central core, 3, of the gum member, 2, such liquid center substantially filling the central core, 3.

The ingredients used to formulate the gum member and the liquid center base are generally well known. In one embodiment the gum member, 2, is made from common sugarfree type chewing gum recipes. The gum member, 2, ingredients may include sorbitol, gum base, mannitol, glycerin, acesulfame potassium, softeners, aspartame, and flavoring. The gum base used to form the gum member, 2, may generally include gum base, elastomers, solvents, emulsifiers, plasticizers, fillers, sweeteners, flavors, coloring agents, antioxidants, preservatives and the like, all of which are known to one skilled in the art. Other conventional chewing gum additives known to one having ordinary skill in the art may also be used in the gum base. In one embodiment, the gum base comprises a dietary supplement. In another embodiment, the gum base comprises an appetite suppressant. In yet another embodiment, the gum base comprises a natural energizer. In still another embodiment, the gum base comprises at least two members of the group consisting of: dietary supplements, appetite suppressants, and natural energizers.

Additionally, the gum base may include crystals (not shown). The composition of such crystals and the addition of such crystals to the gum base are known to one skilled the art. In one embodiment, the crystals comprise a dietary supplement. In another embodiment, the crystals comprise an appetite suppressant. In yet another embodiment, the crystals comprises a natural energizer. In still another embodiment, the crystals comprise at least two members of the group consisting of: dietary supplements, appetite suppressants, and natural energizers.

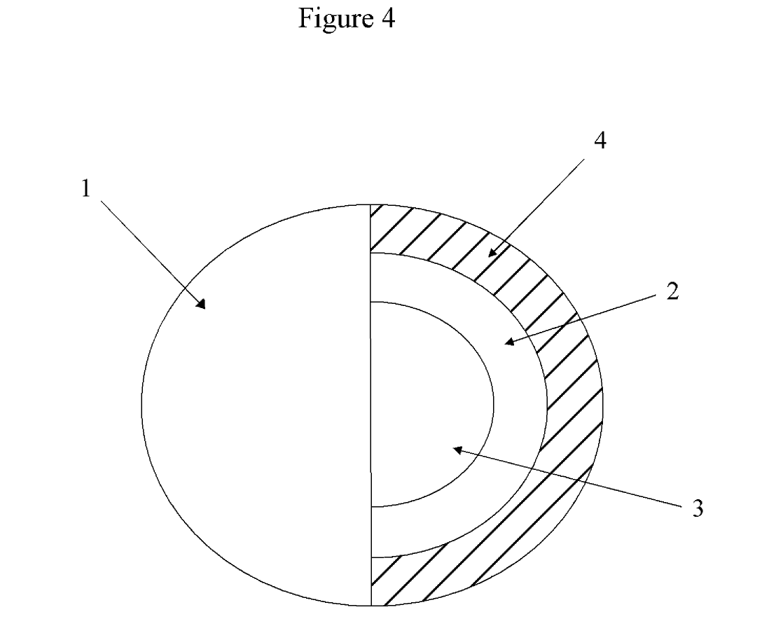

Referring now to FIG. 4, the gum member may include a coating, 4, covering the exterior surface of the gum member. The coating, 4, of gum is known to one skilled in the art. The coating, 4, may be either a powder or a liquid. In one embodiment, the coating, 4, comprises a dietary supplement. In another embodiment, the coating, 4, comprises an appetite suppressant. In yet another embodiment, the coating, 4, comprises a natural energizer. In still another embodiment, the coating, 4, comprises at least two members of the group consisting of: dietary supplements, appetite suppressants, and natural energizers.

The liquid center base may be any liquid center that is known to one skilled in the art, including sugar and sugarless liquid centers. These liquid centers include, without limitation, substantially aqueous centers, aqueous centers comprising additives such as thickeners, emulsifiers, humectants, binders, gums, and the like, and mixtures thereof to prevent migration of the liquid center, suspensions, and semi-liquid centers, wherein the viscosity of the liquid center can range from a gel-like consistency or pasty quality, to a relatively fluid, sticky liquid quality. The liquid center base may comprise traditional ingredients well known in the chewing gum and confectionery arts, such as flavoring agents, sweetening agents, and the like, and mixtures thereof. Additionally, the liquid center may comprise a dietary supplement, an appetite suppressant and/or a natural energizer. In one embodiment, the liquid center comprises a dietary supplement. In another embodiment, the liquid center comprises an appetite suppressant. In yet another embodiment, the liquid center comprises a natural energizer. In still another embodiment, the liquid center comprises at least two members of the group consisting of: dietary supplements, appetite suppressants, and natural energizers.

Any dietary supplement that exhibits healthful benefits in human beings may be used. Dietary supplements that may be used include, but are not limited to: chromium polynicotinate, vitamin B12, caffeine, xanthinol nicotinate, inositol hexaniacinate, green tea (Camilla Sinensis), bitter orange (Citrus Arantium), brown seaweed extract (Fucoidan), acid protease, amylase, cellulose, glucoamylase, lactase, lipase, bromelain, and lactobacillus acidophilus.

The appetite suppressants include commonly known appetite suppressants known to reduce the appetite of the person chewing the chewing gum. A non-exclusive list of appetite suppressants includes: chromium polynicotinate, xanthinol nicotinate, bitter orange (Citrus Arantium), and brown seaweed extract (Fucoidan).

Similarly, the natural energizers include commonly known natural energizers known to increase the energy of the person chewing the chewing gum. Examples of suitable natural energizers include, but are not limited to the following: chromium polynicotinate, vitamin B12, caffeine, and bitter orange (Citrus Arantium).

Chromium polynicotinate has a greater biological activity than other forms of chromium, including sources from picolinate. Chromium polynicotinate stabilizes blood sugar levels and may be critical to the synthesis of cholesterol, fats, and proteins. Also, chromium polynicotinate is essential for energy, promotes loss of fat, and increases lean muscle tissue.

Vitamin B12 is important for the normal functioning of the brain, nervous system, and formation of blood. Additionally, vitamin B12 is involved in metabolism in every cell of the body and promotes energy.

Caffeine is a central nervous system stimulant which promotes energy and mental alterness. Caffeine is also a mild diuretic.

Xanthinol nicotinate is a form of niacin which easily passes through cell membranes. Xanthinol nicotinate increases brain glucose metabolism, improves brain ATP levels and improves blood flow in the brain. Xanthinol nicotinate assists in weight loss and may cause a “flushing” of the skin and a sense of warmth.

Inositol hexaniacinate assists in lowering cholesterol.

Green tea (Camilla Sinensis) is an antioxidant which may assist in treatment of cancer, rheumatoid arthritis, high cholesterol levels, cardiovascular disease, infection, and impaired immune system. Green tea also assists in weight loss.

Bitter orange (Citrus Arantium) is an appetite suppressant which also increases energy.

Brown seaweed extract (Fucoidan) assists in weight loss. Brown seaweed extract also may cause certain rapidly growing cancer cells to self destruct.

In one embodiment, the chewing gum, 1, is comprised of a gum member, 2, and a dietary supplement liquid center, 3. The liquid center comprises both an appetite suppressant and a natural energizer.

In one embodiment the gum member, 2, comprises both an appetite suppressant and a natural energizer. In another embodiment, both the gum member, 2, and the liquid center, 3, comprises an appetite suppressant and a natural energizer. In yet another embodiment, the gum member, 2, may comprise either an appetite suppressant or a natural energizer and the liquid core, 3, comprises the other.

In a further embodiment, the gum member, 2, comprises crystals. The crystals also may comprise an appetite suppressant and/or natural energizer. Additionally, the crystals may be time-release crystals which are also known to one skilled in the art.

In another embodiment, the gum member, 2, is coated with a coating, 4. The coating, 4, may also comprise an appetite suppressant, a natural energizer, or both.

In yet a further embodiment, a method for delivering a dietary supplement, such as an appetite suppressant and/or natural energizer is disclosed. The method consists of a user chewing a piece of chewing gum, 1, which comprises an appetite suppressant and/or a natural energizer. The dietary supplement, such as an appetite suppressant and/or natural energizer are ingested by the user through three different aspects of the chewing gum, 1: the gum coating, the gum member, 2, and the liquid center. The gum member, 2, comprises a gum base, as described herein, which includes an appetite suppressant and/or a natural energizer. The gum base may include crystals which also comprise an appetite suppressant and/or a natural energizer. The crystals may include time-release crystals. The gum member, 2, has a central core, 3, which is substantially filled with a liquid center. The liquid center may either be gel-like or fluid. Additionally, the liquid center comprises an appetite suppressant and/or natural energizer. The exterior of the gum member, 2, is coated with a coating, 4. The coating, 4, also comprises an appetite suppressant and/or a natural energizer.

With respect to the above description, it is to be realized that the optimum dimensional relationship for the parts of the invention, to include variations in size, materials, shape, form, function and manner of operation, assembly and use, are deemed readily apparent and obvious to one skilled in the art, and all equivalent relationships to those described in the specification are intended to be encompassed by the present invention.

Therefore, the foregoing is considered as illustrative only of the principles described herein. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A chewing gum comprising:
a gum member, said gum member encapsulating a central cavity;
a core element located within said central cavity; and
at least one dietary supplement, wherein said at least one dietary supplement is selected from the group consisting of chromium polynicotinate, xanthinol nicotinate, inositol hexaniacinate, bitter orange (Citrus Arantium), brown seaweed extract (Fucoidan), acid protease, cellulose, glucosamylase, lactase, bromelain, and lactobacillus acidophilus.

2. The chewing gum of claim 1, wherein said core element comprises a fluid composition.

3. The chewing gum of claim 1, wherein said core element comprises a semi-fluid composition.

4. The chewing gum of claim 1, wherein said core element comprises a gel-like composition.

5. The chewing gum of claim 1, wherein said dietary supplement is incorporated into said gum member.

6. The chewing gum of claim 1, wherein said dietary supplement is incorporated into said core element.

7. The chewing gum of claim 1, wherein said at least one dietary supplement is incorporated in said core element and said core element comprises a first dietary supplement and a second dietary supplement, said first dietary supplement comprising a natural energizer and said second dietary supplement comprising an appetite suppressant.

8. The chewing gum of claim 1, wherein a coating encapsulates said gum member.

9. The chewing gum of claim 8, wherein said coating comprises a dietary supplement.

10. The chewing gum of claim 9, wherein said dietary supplement comprises an appetite suppressant.

11. The chewing gum of claim 9, wherein said dietary supplement comprises a natural energizer.

12. The chewing gum of claim 1, wherein said gum member comprises crystals.

13. The chewing gum of claim 12, wherein said crystals comprise a dietary supplement.

14. The chewing gum of claim 1, wherein said dietary supplement is incorporated into said core element and said dietary supplement comprises an antioxidant.

15. A chewing gum comprising:
a gum member, said gum member encapsulates a central cavity; and
a core element located within said central cavity, said core element comprising a first dietary supplement and a second dietary supplement and said core element substantially filling said central cavity, wherein said at least one dietary supplement is selected from the group consisting of chromium polynicotinate, xanthinol nicotinate, inositol hexaniacinate, bitter orange (Citrus Arantium), brown seaweed extract (Fucoidan), acid protease, cellulose, glucosamylase, lactase, bromelain, and lactobacillus acidophilus.

16. The chewing gum of claim 15, wherein said first dietary supplement is an appetite suppressant and wherein said second dietary supplement is a natural energizer.

17. The chewing gum of claim 15, wherein said first dietary supplement is a natural energizer and wherein said second dietary supplement is an appetite suppressant.

18. A chewing gum comprising:
a gum member, said gum member encapsulating a central cavity;
a core element located within said central cavity, said core element substantially filling said central cavity, and said core element comprises a gel-like composition;
a coating encapsulating said gum member; and
at least one dietary supplement, wherein said at least one dietary supplement is selected from the group consisting of chromium polynicotinate, xanthinol nicotinate, inositol hexaniacinate, bitter orange (Citrus Arantium), brown seaweed extract (Fucoidan), acid protease, cellulose, glucoamylase, lactase, bromelain, and lactobacillus acidophilus.

19. The chewing gum of claim 18, wherein said core element comprises a first dietary supplement and a second dietary supplement, said first dietary supplement comprising a natural energizer and said second dietary supplement comprising an appetite suppressant.

20. The chewing gum of claim 18, wherein said at least one dietary supplement comprises an antioxidant.
